(19) United States
(12) Patent Application Publication
(10) Pub. No.: US 2019/0254937 A1
(43) Pub. Date: Aug. 22, 2019

(54) Self-Adherent Therapeutic Material

(71) Applicants: Aldo Laghi, Pinellas Park, FL (US); Alps South Europe, S.R.O., Plzen (CZ)

(72) Inventors: Aldo Laghi, Pinellas Park, FL (US); Nathaniel Vint, Palm Harbor, FL (US)

(73) Assignee: Alps South Europe, S.R.O., Plzen (CZ)

(21) Appl. No.: 16/402,877

(22) Filed: May 3, 2019

Related U.S. Application Data
(63) Continuation-in-part of application No. 16/382,582, filed on Apr. 12, 2019.
Application No. 16/382,582 is a continuation-in-part of application No. 16/008,525, filed on Jun. 14, 2018.
Application No. 16/008,525 is a continuation-in-part of application No. 15/656,564, filed on Jul. 21, 2017.
Application No. 15/656,564 is a continuation-in-part of application No. 14/602,893, filed on Jan. 22, 2015.
(60) Provisional application No. 62/658,059, filed on Apr. 16, 2018.
(60) Provisional application No. 62/062,372, filed on Oct. 10, 2014.
(60) Provisional application No. 62/051,847, filed on Sep. 17, 2014.
(60) Provisional application No. 62/040,771, filed on Aug. 22, 2014.

(51) Int. Cl. A61K 8/02; A61K 8/90; A61K 8/92; A61Q 19/00

(52) U.S. Cl. CPC A61K 8/0212; A61K 8/90; A45D 44/002; A61Q 19/00; A61K 8/922

(57) ABSTRACT

A self-adhering material for use with cosmetic or therapeutic agents. The self-adhering material uses a triblock copolymer in combination with a plasticizing oil having a viscosity between 30-300 centistokes to maintain shape and structure on a user's skin without leaving residue. The self-adhering material may have a fabric backing and may also be embedded with light reflecting materials.

### CROSS-REFERENCE TO RELATED APPLICATIONS

This application is a continuation-in-part of U.S. application Ser. No. 16/382,582 entitled “Phase Change Material for Medical and Cosmetic Applications” which is a continuation-in-part of U.S. application Ser. No. 15/656,564 entitled “Phase Change Material for Thermal Therapy and Delivery of Active Ingredients” filed on Jul. 21, 2017, which claims priority to U.S. Provisional Patent No. 62/364,998 filed on Jul. 21, 2016, and is a continuation-in-part of U.S. application Ser. No. 14/602,893 filed on Jan. 22, 2015, which claims priority to U.S. Provisional Patent Application No. 62/040,771 filed on Aug. 22, 2014, U.S. Provisional Patent Application No. 62/051,847 filed on Sep. 17, 2014, and U.S. Provisional Patent Application No. 62/062,372 filed on Oct. 10, 2014. This application also claims priority to pending provisional application No. 62/658,059 filed Apr. 16, 2018 entitled “Phase Change Material for Reduction of Swelling, Edema, and Bruising.” This application also claims priority to U.S. application Ser. No. 16/008,285 filed on Jun. 14, 2018 entitled “Thermoformable Retainer for Facemasks,” which claims priority to U.S. Provisional Patent Application No. 62/619,936. The contents of each of the above-listed applications are hereby incorporated by reference herein in their entirety.

### BACKGROUND OF THE INVENTION

### Field of the Invention

This invention relates to a cosmetic and therapeutic material. More specifically, this invention relates to a self-adherent lipophilic material which improves dermal penetration of cosmetic or therapeutic ingredients using plasticizers having a high viscosity in combination with polymers.

### Description of the Background Art

Cosmetic and therapeutic patches for the eyes, face, and other body parts are well known in the industry. Most of the masks and patches in commerce are hydrophilic which means that any therapeutic or cosmetic ingredient included with the mask or patch must also be hydrophilic. This means the ingredient is not likely to penetrate the epidermis as skin is generally impermeable to water.

The polymers useful in forming the gel compositions of the invention comprises high viscosity triblock and branched copolymers. The triblock copolymers have the general configuration A-B-A wherein each A is a glassy polymer end block segment of polystyrene and B is a elastomeric polymer center block segment of poly(ethylene-butylene), poly(ethylene-propylene) or poly(ethylene-ethylene-propylene). The useful high viscosity branched copolymers have the general configuration (A-B)n wherein A is polystyrene and B is (ethylene-butylene), (ethylene-propylene) or (ethylene-ethylene-propylene) and the subscript n is an number. The B and A portions of the triblock and branched copolymers are incompatible and form a two-phase system consisting of sub-micron domains of glassy polystyrene interconnected by flexible B chains. These domains serve to crosslink and reinforce the structure. This physical elastomeric network structure is reversible, and heating the polymer above the softening point of polystyrene temporarily disrupt the structure, which can be restored by lowering the temperature.

In a prior invention, a thermoplastic elastomer comprising a triblock copolymer was disclosed and is reflected in U.S. application Ser. No. 14/602,894 (“'894 application”) entitled “Post-Surgical Articles for Reduction of Swelling, Edema, and Bruising,” filed on Jan. 22, 2015, and is hereby incorporated by reference herein. The disclosed invention in the '894 application provided a means of warm thermal therapy for various types of post-surgical procedures for reducing the negative after-effects of an operation, such as edema and swelling. The invention was able to achieve this by utilizing the conformability, durability, and low thermal conductivity of a phase change material situated within an elastomeric matrix, as compared to other hydro gel and water-based substances.

Therefore, it is an object of this invention to provide an improvement which overcomes the aforementioned inadequacies of the prior art devices and provides an improvement which is a significant contribution to the advancement of phase change materials.

It is a further object of this invention to provide a material which can be applied to the skin which leaves no residue.

It is a further object of this invention to provide a material which can deliver cosmetic or therapeutic ingredients through the epidermis.

It is a further object of this invention to provide a material which is comfortable on the skin.

It is a further object of this invention to provide a material that does not require an impermeable barrier such as a bag.

The foregoing has outlined some of the pertinent objects of the invention. These objects should be construed to be merely illustrative of some of the more prominent features and applications of the intended invention. Many other beneficial results can be attained by applying the disclosed invention in a different manner or modifying the invention within the scope of the disclosure. Accordingly, other objects and a fuller understanding of the invention may be had by referring to the summary of the invention and the detailed description of the preferred embodiment in addition to the scope of the invention defined by the claims taken in conjunction with the accompanying drawings.

### SUMMARY OF THE INVENTION

For the purpose of summarizing this invention, this invention discloses a material comprising a triblock copolymer and a plasticizer of a certain viscosity such that the material self-adheres to a user's skin. The lipophilic tendencies of the material disclosed allows for the penetration of the epidermis via the sebaceous tracts by therapeutic or cosmetic ingredients. Preferably, the material comprises a triblock copolymer in an amount between 2-30% and a plasticizing oil between 35-98%. The material can also incorporate therapeutic or cosmetic agents in an amount between 0.1% and 35%. The material can be combined with retainers or fabrics for further ease of use.

The foregoing has outlined rather broadly the more pertinent and important features of the present invention in order that the detailed description of the invention that follows may be better understood so that the present contribution to the art can be more fully appreciated. Additional features of the invention will be described hereinafter which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a more complete understanding of the present disclosure and its advantages, reference is now made to the following descriptions, taken in conjunction with the accompanying drawings, in which:

FIG. 1 depicts a front view of the phase change material incorporated into, for example, a post-surgical treatment garment as dictated by a user having a facial injury or as therapeutic aid after a chemical peel or dermabrasion or cosmetic facial treatment;

FIG. 2 depicts a cross section of the layers included within the phase change material incorporated into, for example, a post-surgical treatment garment as dictated by a user having a facial injury;

FIG. 3 depicts an exploded view of the layers included within the phase change material incorporated into, for example, a post-surgical treatment garment as dictated by a user having a facial injury;

FIG. 4 depicts a back view of the phase change material incorporated into, for example, a post-surgical treatment garment as dictated by a user having a facial injury;

FIG. 5 depicts a front view of the phase change material incorporated into, for example, a post-surgical treatment garment as dictated by a user having a facial injury;

FIG. 6 depicts a front view of the phase change material incorporated into, for example, a post-surgical treatment garment as dictated by a user having a facial injury;

FIG. 7 depicts a back view of the phase change material incorporated into, for example, a post-surgical treatment garment as dictated by a user having a facial injury;

FIG. 8 depicts a front view of the phase change material incorporated into, for example, a post-surgical treatment garment as dictated by a user having a facial injury;

FIGS. 9(a) and 9(b) depict multiple side views of the phase change material incorporated into, for example, a post-surgical treatment garment as dictated by a user having a facial injury;

FIG. 10 depicts a garment according to another embodiment including the phase change material permanently positioned on an elongate, elastic bandage and adapted for application to a wearer's abdominal area;

FIGS. 11 and 12 depict multiple views of a garment according to another embodiment including the phase change material permanently positioned on an elongate, elastic bandage and adapted for application to a wearer's chin;

FIG. 13 depicts another embodiment including a phase change material adapted to conform to a wearer's breast;

FIG. 14 depicts a top view of another embodiment of the phase change material incorporated into, for example, a post-surgical treatment garment as dictated by a user having a facial injury;

FIG. 15 depicts a perspective view of the phase change material incorporated into, for example, a post-surgical treatment garment as dictated by a user having a facial injury;

FIGS. 16(a) and 16(b) depict another embodiment of the phase change material incorporated into, for example, a post-surgical treatment garment as dictated by a user having a facial injury; FIG. 16(b) further depicts a cross section of an embodiment showing the knitted outer layer, the thermoformable resin layer, and the phase change material positioned on the thermoformable resin layer;

FIGS. 17(a) and 17(b) depicts various perspective views of the phase change material incorporated into, for example, a post-surgical treatment garment as dictated by a user having a facial injury;

FIG. 18 depicts a garment including the phase change material incorporated into, for example, a post-surgical treatment garment as dictated by a user having a chin or neck injury.

FIG. 19 is a perspective view of a two-piece embodiment of the present invention.

FIGS. 20 and 21 are perspective views of another two-piece embodiment of the present invention in which the thermoformable layer has a bonded layer of fabric.

FIG. 21A is a section view of the embodiment shown in FIG. 21.

FIGS. 22 and 23 are perspective views of one-piece embodiment of the present invention in which the strapped fabric is bonded to the thermoformable layer.

FIG. 23A is a section view of the embodiment shown in FIG. 23.

FIGS. 24 and 25 are perspective views of a one-piece embodiment of the present invention in which the thermoformable layer has one layer of strapped fabric bonded to one side and one layer of strapless fabric bonded to the other side.

FIG. 25A is a section view of the embodiment shown in FIG. 25.

FIGS. 26 and 27 are perspective views of a one-piece embodiment of the present invention in which the thermoformable layer is bonded to the strapped fabric on one side and strapless fabric is bonded onto the other side. In this embodiment the cosmetic or therapeutic mask is affixed to the other side of the strapless fabric.

FIG. 27A is a section view of an embodiment shown in FIG. 27.

FIG. 28 depicts a thermally assisted therapeutic aid, in the shape of a facial mask, for cosmetic and wound treatments in an exploded view and in composite form.

FIG. 29 depicts the phase change material.

FIG. 30 depicts a thermally assisted therapeutic aid, without regard to a specific shape, for cosmetic and wound treatments in an exploded view and in composite form.

FIG. 31 is a table showing the force required to separate the invention from a substrate using the 180° test when using a high molecular weight polymer.

FIG. 32 is a graph showing the force required to separate the invention from a substrate as a function of plasticizing oil viscosity when using high molecular weight polymer.

FIG. 33 is a table showing the force required to separate the invention from a substrate using the 180° test when using a low molecular weight polymer.

FIG. 34 is a graph showing the force required to separate the invention from a substrate as a function of plasticizing oil viscosity when using low molecular weight polymer.

### DETAILED DESCRIPTION OF THE PREFERRED EMBODIMENT

The following description is of the best mode presently contemplated for carrying out the invention. This description is not to be taken in a limiting sense, but is made merely for the purpose of describing one or more preferred embodiments of the invention. The scope of the invention should be determined with reference to the claims.

In certain embodiments, thermoplastic elastomers according to the present invention comprise a styrenic block copolymer that is a non-hydrogenated styrenic block copolymer such as poly(styrene-butadiene-styrene) (SBS), poly(styrene-isoprene-styrene) (SIS), or poly(styrene-isoprene-butadiene-styrene) (SIBS), or a hydrogenated styrenic block copolymer such as poly(styrene-ethylene-butylene-styrene) (SEBS), poly(styrene-ethylene-propylene-styrene) (SEPS), or poly(styrene-ethylene-ethylene-propylene-styrene) (SEEPS), or combinations thereof. Preferably, the present invention uses SEEPS but may use any suitable styrenic block copolymer having the appropriate stretch and stiffness characteristics. In certain aspects, the triblock copolymer is one with the trade names of Septon and Kraton and may be included within the phase change material at a range from 2-30% by weight. Preferably, the triblock copolymer is provided in a range of 4-10% by weight. The amount of triblock copolymer used is dependent on the specific polymer used in the matrix, the degree of stiffness desired when the temperature of the garment is lowered, and the specific PCM used.

In certain aspects, the disclosed phase change material is made with and/or includes one or more plasticizing oils such as mineral oil, white oil, paraffinic oil, naphthenic oil, vegetable oil, or a synthetic liquid oligomer such as a polybutene, a polypropylene, or a polyterpene oil. In certain aspects, the oil may also be seeded with an insoluble fine powder such as talc. Brand name products such as Hydrobrite 1000 can also be used. The oil or other plasticizing agent is preferably within the range of approximately 0-70% by weight with a preferred range of 40-60% by weight when used with the low temperature phase change materials. Alternatively, the oil or other plasticizing agent is preferably within the range of 30-80% by weight with a preferred range of 40-60% by weight when used with the higher temperature phase change materials. The plasticizing oil preferably has a viscosity between 5 and 20 centistokes for non-sticky formulations and between 50 and 300 centistokes for those formulations intended to promote a certain stickiness so that the garment maintains position on the user with or without straps.

The phenolic antioxidant may include at least one of isobutylenated methylstyrenated phenol, a styrenated phenol, various o-, m-, p-cresols (e.g., 4,4′thiobis-6-(t-butyl-m-cresol), 4,4′-butylidenebis-b-(t-butyl-m-cresol)), 2,6-di-tert-butyl-p-cresol, (octadecanoxycarbonylether) phenol, tetrakis-(methylene-(3,5-ditertbuty-4-hydrocinnamate)methane, 2,2′-methylenebis (4-methyl-6-nonyl) phenol, 1, 3, 5-tris (3,5-di-tert-butyl-4-hydroxybenxyl-)-1,3,5-triazine-2,4,6 (1H, 3H, 5H)-trione, or any combination thereof. The antimicrobial agents may include, for example, at least one of silver zeolite, silver zirconium phosphate, silver nitrate, silver thiosulfate, silver sulphadiazine, silver fusidate, and quaternary ammonium compounds (“QAC”). Other classes of silver-based antimicrobial agents may be used as well, for example a silver acetate, a silver bromide, a silver carbonate, a silver chlorate, a silver chloride, a silver citrate, a silver fluoride, a silver iodate, a silver lactate, a silver nitrate, a silver nitrite, a silver perchlorate or a silver sulfide. The antioxidant and/or antimicrobial additives may be added in the rage of 0-10% by weight, preferably 0-1%.

In alternative aspects, one or more of the above discussed additives may be added to the mixture after the mixture is melted or during the cooling process. After heating and mixing the mixture of styrene-based polymer, the plasticizing oil(s), and one or more optional additives, these components are melted together in such a manner that a homogeneous, molten mixture is obtained. Specifically, one method of manufacture involves mixing the ingredients and bringing the mixture to a melting temperature between 350° F. and 450° F. After obtaining the molten mixture, the molten mixture may be extruded via an extruder, molded via a molding machine, or injected into a closed mold, open mold, or other similar vessel into the desired shape. Upon final formation, the ingredients form a gel matrix.

Within the thermoplastic elastomer is a phase change material. When lower temperatures are used, the phase change material is a lipid or a combination or association of one or more ligands that change phase from solid to liquid at a low temperature. The phase change lipid or materials are of the hydrocarbon type such as N-alkenes or N-alkanes. Applicable examples are 1-octene, 1-decene, 1-dodecene, 1-tridecane, 1-tetradecene, etc. or linear alpha olefins such as those marketed by Chevron Philips and Ineos with carbon numbers ranging between 8 and 18. In one embodiment, the phase change lipids and/or materials are composed of tetradecene, which has 14 carbon molecules and utilizes a double bond on the first carbon. Further, it was found that any carbon chain with proper vapor pressure and in the proper melting point range would be sufficient. Additionally, other hydrocarbons, alcohols, esters, or vegetable oils having a phase change temperature between −10° C. to 25° C. and preferably between 0° C. to 20° C. may be used. Any of the described phase change materials may be used in combination with each other or individually within the matrix described herein.

When higher temperatures are desired, the phase change material is a paraffin or linear alpha olefin having a fusion temperature between 45° C. to 65° C. Appropriate examples include those linear alpha olefins marketing by Chevron Phillips and Ineos with carbon numbers of 24 to 30 and mixtures thereof. Other suitable materials include hydrocarbons, alcohols, esters, hydrogenated and non-hydrogenated vegetable oils having a phase change temperature between 45° C. to 65° C.

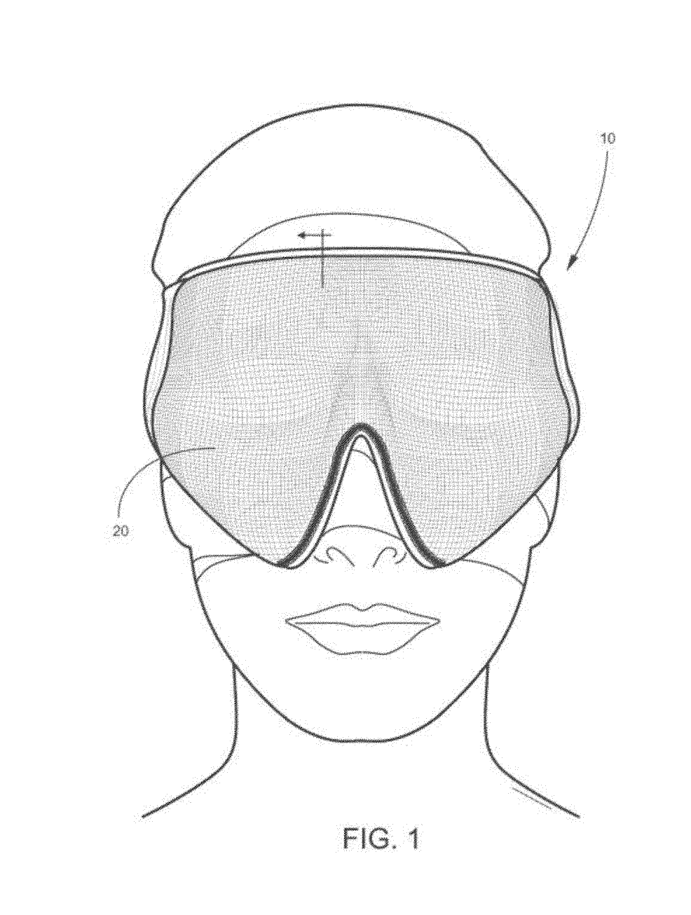

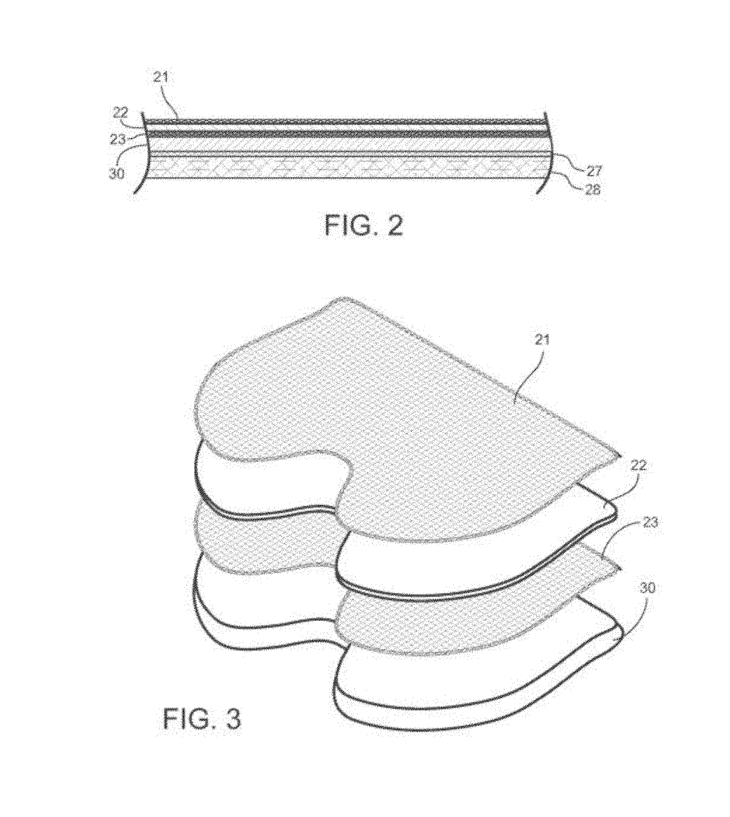

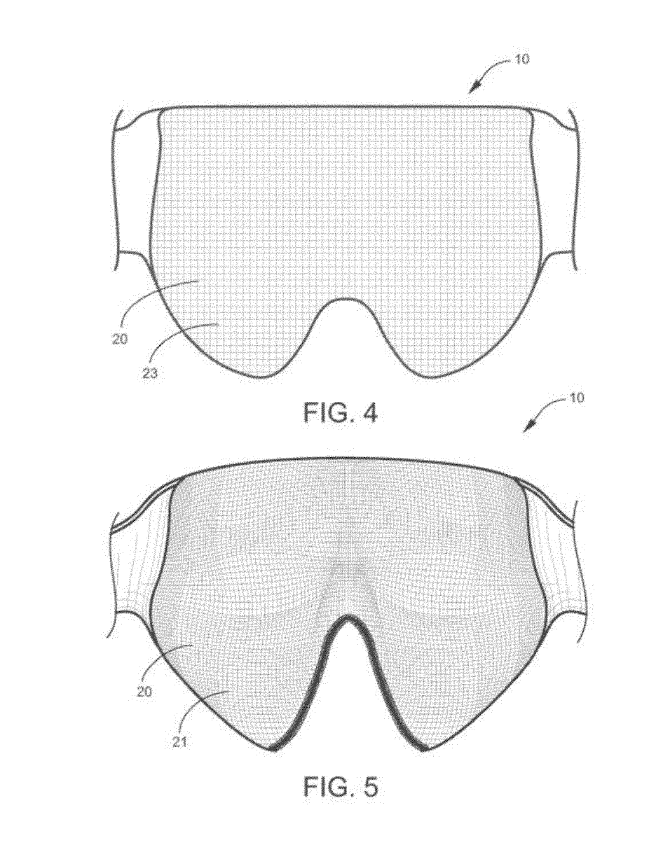

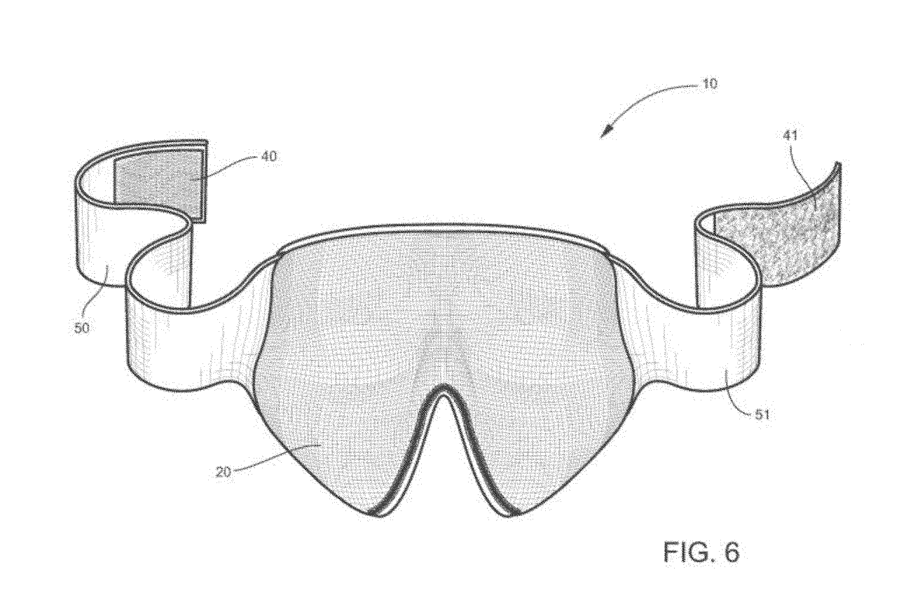

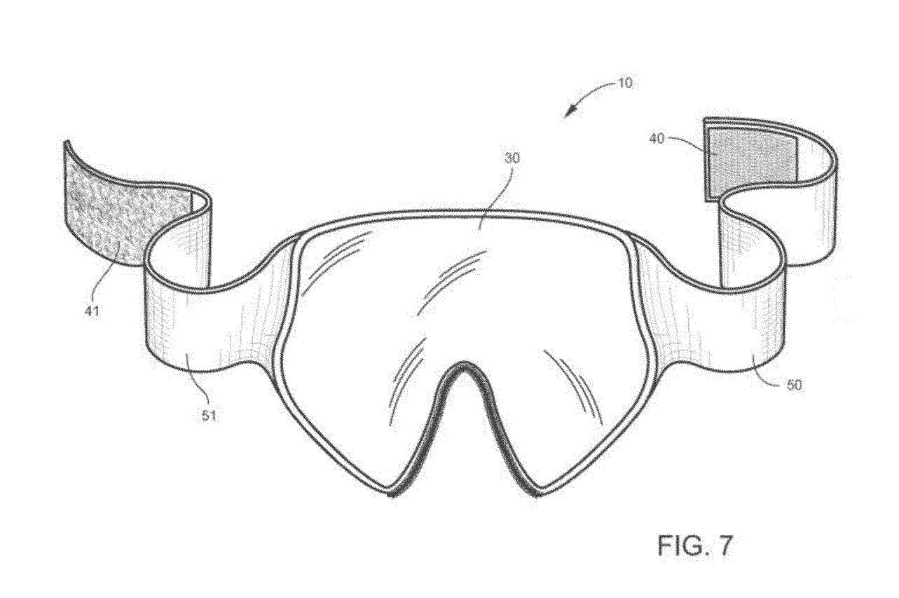

The disclosed garments will now be described in more detail with reference to the Figures FIGS. 1-7 disclose a phase change material incorporated into, for example, a garment 10 as dictated by a user having a facial injury according to a first embodiment including a thermoformable assembly 20 comprising a thermoformable resin 22 arranged between inner 23 and outer 21 knitted fabric layers, the thermoformable assembly 20 configured to be heated to an appropriate temperature of the thermoformable resin 22 such that the thermoformable assembly 20 can be shaped to conform to contours of a user's face. In certain aspects, inner layer 23 is optional and may not be present. In this aspect, the garment 10 also includes a styrene-based polymeric gel layer 30 arranged on the inner knitted fabric layer 23, or when inner knitted fabric layer 23 is not present, gel layer 30 may be arranged directly on thermoformable resin 22. As depicted in FIG. 2, when in use, gel matrix layer 30 of garment 10 can be placed on bandage 27, which directly covers flesh and/or post-surgical site 28. However, in certain aspects, the gel matrix layer 30 is configured to directly contact the wearer's skin/flesh.

In certain aspects, the inner and outer layer knitted fabric layers 21, 23 are selected to further synergistically maximize the reduction and/or prevention of post-surgical, excessive bruising, swelling, and edema associated with surgical procedures. The inner fabric layer 21 and outer fabric layer 23 are optional for the garment 10 with the addition of either layer being included for support. For example, these knitted fabric layers and the yarns included in the fabric layers and the filaments included within the yarns of the knitted layers may be selected to maximize compressibility of the gel to further maximize post-surgical treatment for the reduction and/or prevention of bruising, swelling, and edema. Depending on the desired effects, the inner and outer knitted fabric layer are the same, or alternatively, the inner and outer knitted fabric layer are different. For example, the knitted inner and outer layer fabric layers may independently be made from, for example, a non-low melt polyester yarn, a non-low nylon yarn, a non-low polypropylene yarn, a non-low melt polyethylene yarn, cotton yarn, wool yarn, any combinations thereof, and these yarns may be either multifilament or monofilament. In certain aspects, the yarns included within the inner and outer layer knitted fabric layers are multifilament having a mass ranging from 110 to 160 denier, 120 to 150 denier, 130 to 145 denier, and 135 to 145 denier. In certain aspects, the knit fabric of the inner and outer fabric layers are independently a weft knit having multidirectional stretch characteristics that aid in further enhancing durability of the thermoformable assembly. The knit fabric of the inner and outer fabric layers may independently include from 6 to 12 courses per cm², more preferably from 8 to 10 courses per cm and from 9 to 14 wales per cm², more preferably 10 to 12 wales per cm² in the weft knit. In certain preferred aspects, the inner and outer fabric layers independently include 9 courses and 11 wales per cm². In certain aspects, the knit fabric of the inner and outer fabric layers independently have an elasticity ranging from 80 to 140%, preferably 100 to 130%, and most preferably 115 to 125% in a vertical direction and from 60 to 100%, preferably 70 to 90%, and most preferably 75 to 85% in the horizontal direction. In certain aspects, the inner layer 21, outer layer 23, and the knitted body configured to be the thermoformable resin may independently include a milano knitting pattern, a plain jersey knitting pattern, an interlock jersey knitting pattern, an interlock knitting pattern, a rib knitting pattern, a ponte de roma knitting pattern, or any combination thereof.

When initially making the thermoformable assembly, the knitted body made from a co-polyester yarn, a poly-caprolactone yarn, or a combination thereof and optionally having a polyester fiber may be positioned in between the inner and outer layer fabric layers. After layering this stack, this layered stack is heated to a sufficient temperature to melt the knitted body from a co-polyester yarn, a poly-caprolactone yarn, or a combination thereof to bond these layers together. In certain aspects and because the knitted body is made from a co-polyester yarn, a poly-caprolactone yarn, or a combination thereof includes fabric “windows”, this knitted body of co-polyester yarn, a poly-caprolactone yarn, or a combination thereof may be preferred over a solid sheet (e.g., a laminate layer) made from the same material because the windows and knitted structure may allow for stronger bonding while concurrently lowering manufacturing costs of the thermoformable assembly. However, in certain alternative aspects, a solid sheet of the thermoformable resin may be used when manufacturing the thermoformable assembly. In additional aspects, the thermoformable assembly is preferably configured to be repeatedly heated and reshaped to the user's contours. For example, with regard to a rhinoplasty, the thermoformable assembly may be heated to or slightly above the thermoformable resin's transition temperature to ensure that the thermoformable assembly may be shaped to the user's contours. It is important that thermoformable resin hardens and becomes rigid after heating to (or beyond) its transition temperature. However, it is also important that the thermoformable resin does not become brittle after heating such that the thermoformable assembly is fragile, lacks durability, and may not be re-heated and/or re-shaped. Thus, the disclosed thermoformable assembly may be advantageously repeatedly heated and re-shaped into a rigid structure having desired contours.

The garment 10 may include a strap 50, 51 configured to secure around a user's head to hold the garment 10 in place on the user's face, and the garment 10 includes hook and loop fasteners 40, 41 for securing the strap to the garment 10. In certain aspects, the hook or loop fastener may be attached to one end of the strap and the complimentary hook or loop fastener may be attached on the thermoformable assembly 20 or on another strap. For example, in certain aspects, the garment 10 includes two straps 50, 51 configured to secure around a user's head to hold the garment 10 in place on the user's face.

The garment 10 may preferably be a partial face garment configured for placement over a user's eyes and the bridge of the nose. The styrene based gel of this partial face garment 10 can be heated and/or cooled to a desired temperature and applied and fastened to the user's face to treat post-surgical bruising, swelling, and edema associated with, for example, rhinoplasty, eyelid surgery, check implantation, or any combination thereof. The garment 10 can also be used after dermatological procedures and facial treatments at spas, including procedures such as chemical peels and dermabrasion. This garment 10 is preferred over conventional hydrogel garments because unlike hydrogel based garments, this garment exhibits low thermal conductivity and high durability and resiliency coupled with the ability to provide evenly distributed compressive forces (e.g., a high modulus of elasticity). Additionally, this garment 10 advantageously results in better patient comfort and overall improved healing due to the combination of any of (i) low thermal conductivity, (ii) high durability and resiliency, (iii) evenly distributed compressive forces, and (iv) customizability or formability achieved by post-surgical treatment garment 10. In certain aspects, the garment 10 further includes, for example, fabric or a fabric layer that lines the peripheral edges of garment 10 and conceals one or more layers of the thermoformable assembly. For example, in certain aspects, the fabric or fabric layer completely lines the peripheral edges of garment 10 and completely conceals the thermoformable assembly such that the thermoformable assembly is not visible to the wearer or another observer. In certain aspects, garment 10 is more aesthetically pleasing when the fabric or fabric layer lines the peripheral edges of garment 10.

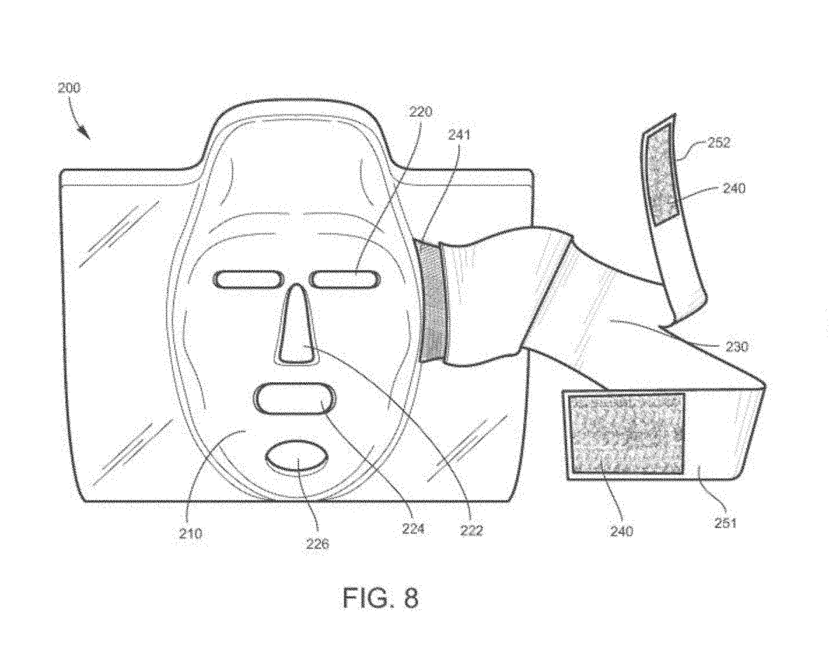

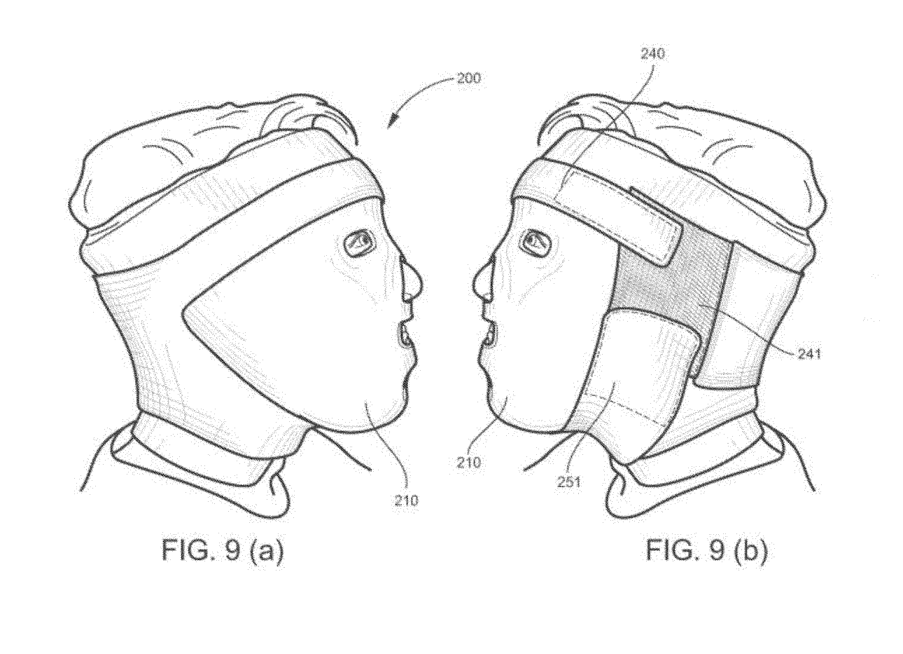

In another embodiment and as further illustrated in FIGS. 8, 9(a), and 9(b), the garment may include a post-surgical face garment 200 including a garment 210 made from a styrene-based gel shaped to conform to and cover a wearer's face including a chin, a nose, cheeks, forehead, and ears, the garment having openings at the wearer's eyes 220, nose 220, mouth 224, and optionally the chin 226; and a strap 230 configured to fasten around the forehead and under the chin to secure the garment in place. For example, this post-surgical face garment 200 may consist essentially of or consist only of the styrene-based gel, which conforms to and covers a wearer's face including a chin, a nose, cheeks, forehead, and ears, the garment having openings at the wearer's eyes, nose, and mouth, and a strap 230 configured to fasten around the forehead and under the chin to secure the garment in place. In certain aspects, the strap includes complimentary hook and look fasteners 240, 241 on opposite ends of the strap. One end of the strap may be bifurcated such that a portion of the bifurcated strap 251 wraps around the chin of a user while the other portion of the bifurcated strap 252 is configured to wrap around the head of the user in such a manner that the garment may be securely fastened to a user with the strap. In certain aspects, the strap is an elastic strap configured to stretch and apply compressive forces when securely fastening the garment to the user. As illustrated in FIGS. 8, 9(a), and 9(b) the strap 230 may be attached to, for example, on the outermost surface of the gel, or in certain aspects, the strap 230 may be positioned within the gel.

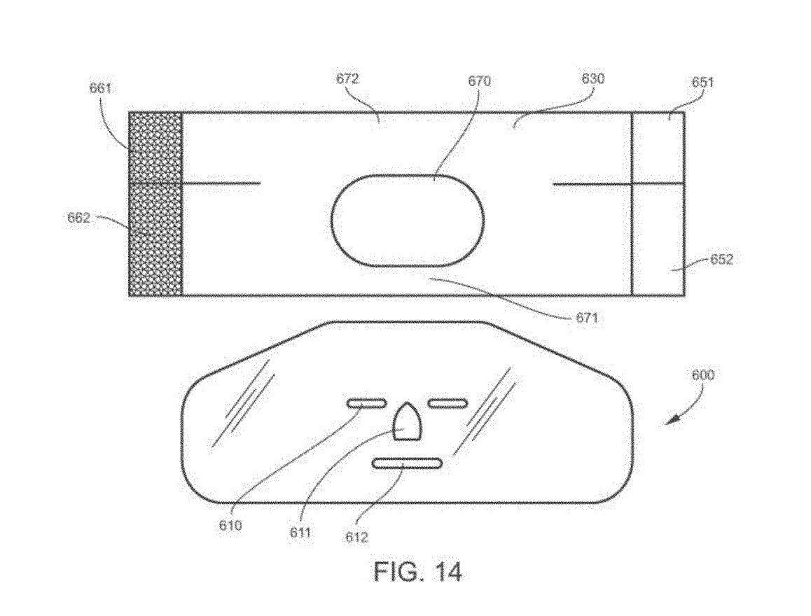

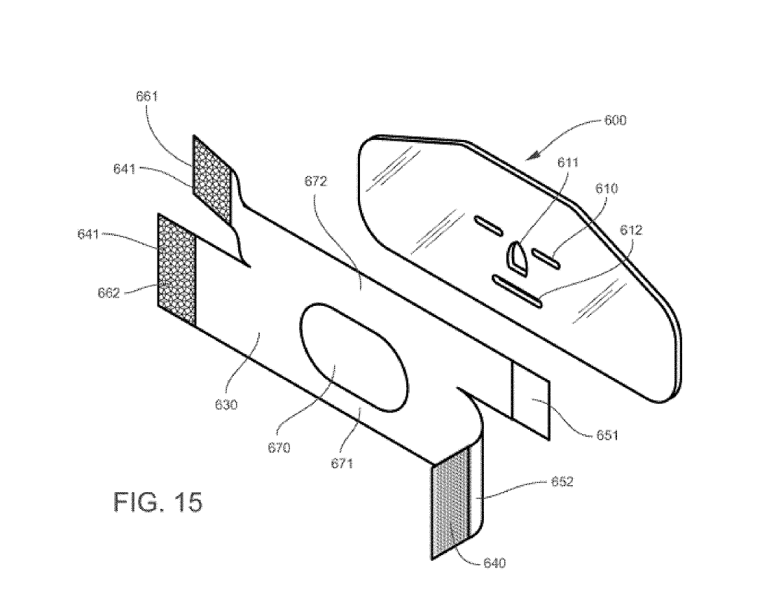

As further illustrated in FIGS. 14 and 15, in certain aspects, a further embodiment of the garment 630 may have various alternative shapes and configurations. For example, the garment 630 may be a strap that is bifurcated at both ends 651, 652, 661, 662. These bifurcated ends 651, 652, 661, 662 may further include complementary hook 640 and loop 641 fasteners. As further illustrated in FIGS. 14 and 15, the garment 630 may include a hole 670 positioned mid-span along the length of the strap 630 such that portions 671, 672 of the strap are configured to concurrently wrap around a wearer's chin and forehead respectively while not contacting and/or obstructing any of the wearer's nose, eyes, mouth, and combinations thereof. The garment depicted in FIGS. 14 and 15 may also include a styrene based gel garment 600 that is removable or permanently attached to strap 630. In certain aspects, the styrene based gel garment 600 and the strap 630 are two separate components that are not permanently attached to one another. In this aspect, the styrene based gel garment 600 may further include eyeholes 610, a nosehole 611, and mouth hole 612 such that the garment 600 can be placed on, for example, the wearer's face, and then the strap 630 including the hole can be positioned over the styrene based gel garment to secure the garment to the wearer. For example, the strap 630 may be placed on the wearer such that portions 672, 671 of the strap around the hole 670 secure the forehead and chin portions respectively of the styrene based gel to the wearer and then the strap may be fastened by the hook and loop fasteners positioned on the bifurcated ends 651, 652, 661, 662. Of note, the garment 630 does not require a fabric layer in order to perform the desired therapeutic function described herein.

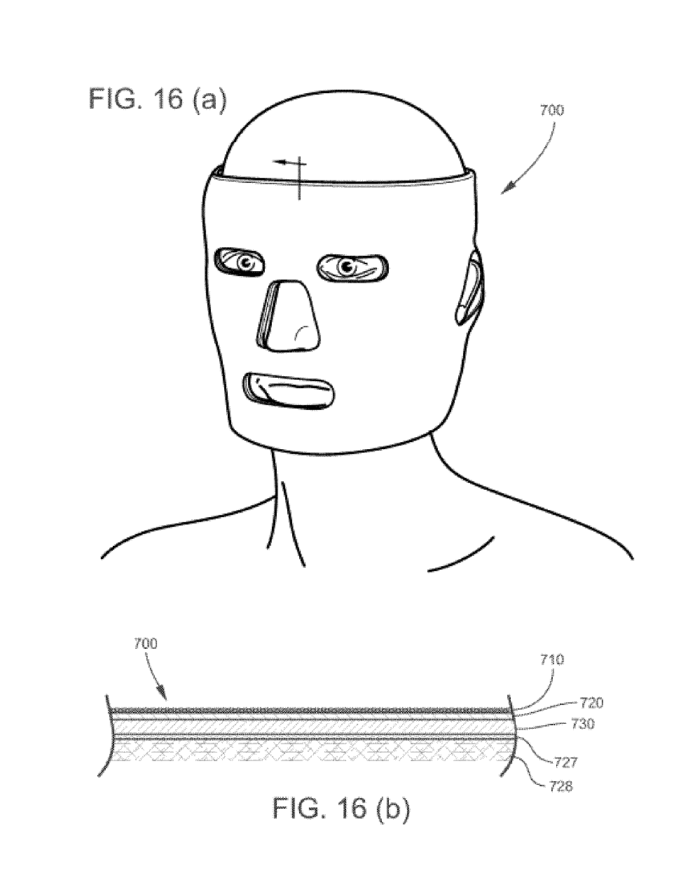

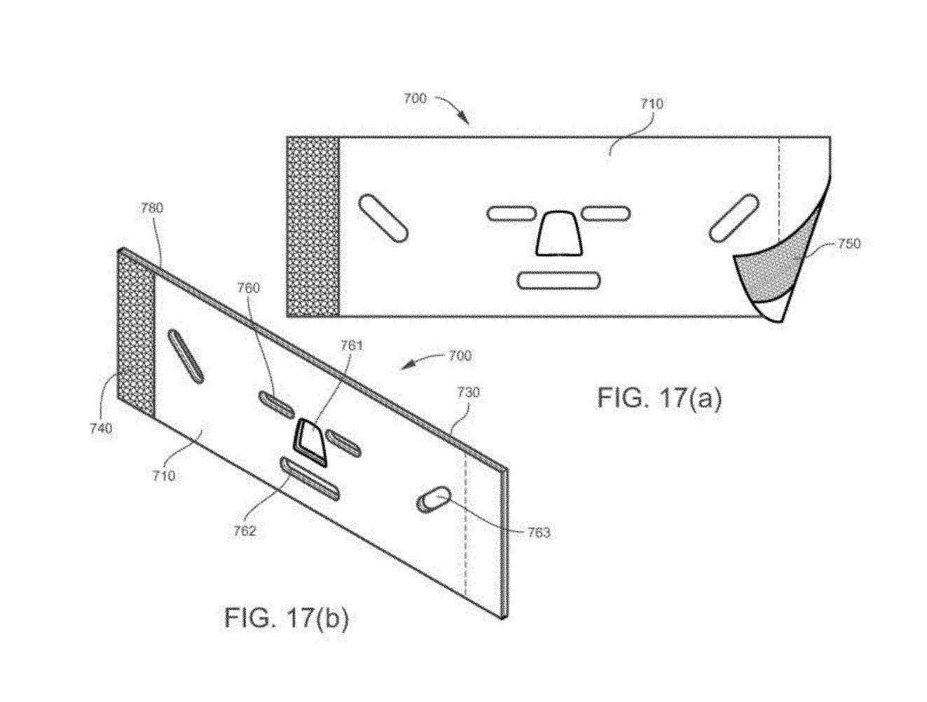

FIG. 16(a), 16(b), 17(a), and 17(b) depict another embodiment of the garment 700 in which the above discussed thermoformable assembly is included on or within an elongate strap 710 and is configured to apply compression. The strap 710 preferably includes sufficient elasticity to stretch over portions of the wearer's head (e.g., completely around the wearer's head), and the strap further preferably includes hook and loop fasteners 740, 750 positioned at opposite ends of the strap that preferably span the entire width the elongate strap 710. The elasticity of elongate strap 710 coupled with the hook and loop fastener widths allow for the compression garment to be properly secured to the wearer's head while concurrently ensuring maximum compression when compared to other conventional designs that lack elasticity and/or hook and loop fasteners having the above discussed features. Also, in this aspect, this garment 700 includes an outer knitted fabric layer 710 (i.e., portion of the elongate strap or the entire elongate strap) as disclosed above, an optional thermoformable resin 720 as disclosed above, and a gel matrix layer 730 as disclosed above that is either detachably positioned or permanently positioned directly on the thermoformable resin 720. In certain aspects, this garment 700 includes eyeholes 760, a nosehole 761, a mouth hole 762, and optionally earholes 763. The styrene based gel 730 of this garment may be adapted to cover portions of the wearer's cheeks, chin, and forehead, and in some aspects, the gel matrix layer 730 of this garment is adapted to additionally substantially cover portions of the wearer's head immediately adjacent either partially or completely around the wearer's ears. Similarly, the thermoformable resin 720 may also be adapted to cover portions of the wearer's cheeks, chin, and forehead, and in some aspects, the styrene based gel of this garment is adapted to additionally substantially cover portions of the wearer's head immediately adjacent either partially or completely around the wearer's ears. To maximize compression therapy results using this garment, the garment may be initially heated (e.g., to the thermoformable resin's transition temperature) as described above and the shaped to the contours of the wearer's face. After shaping this garment to the contours of the wearer's face, the garment may be allowed to harden to achieve a rigidly shaped structure and then subsequently secured onto the wearer's face using the hook and loop fasteners. If cold compression therapy or thermal therapy is further desired, the shaped garment may be chilled or heated to a sufficient temperature and the chilled or heated garment may be subsequently secured on the wearer's face. FIG. 16(b), depicts garment 700 in use in which gel matrix layer 730 is placed on bandage 727, which directly covers flesh and/or post-surgical site 728.

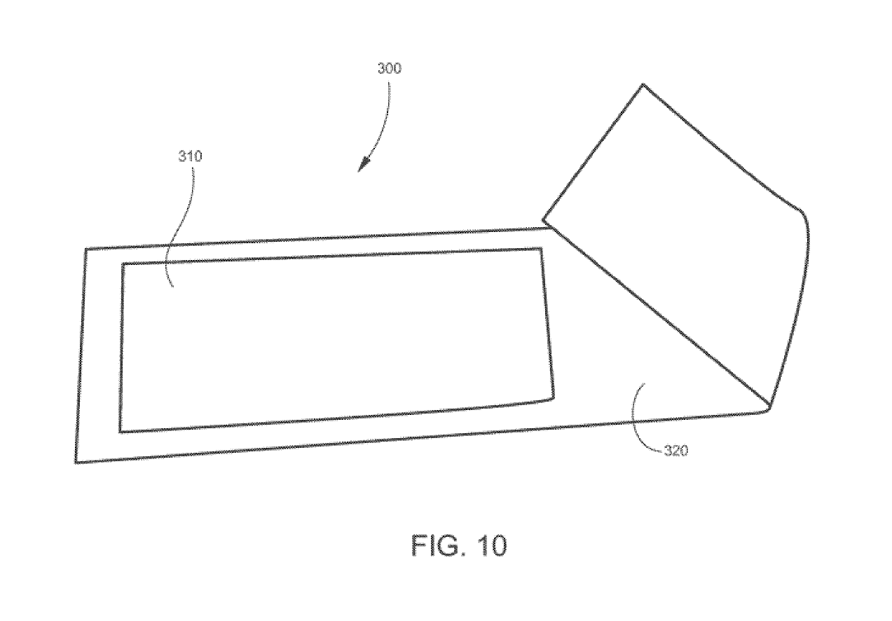

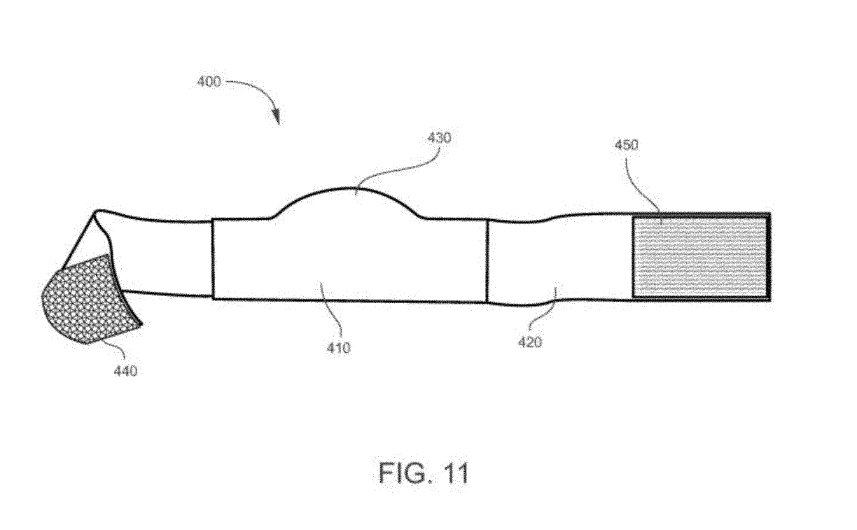

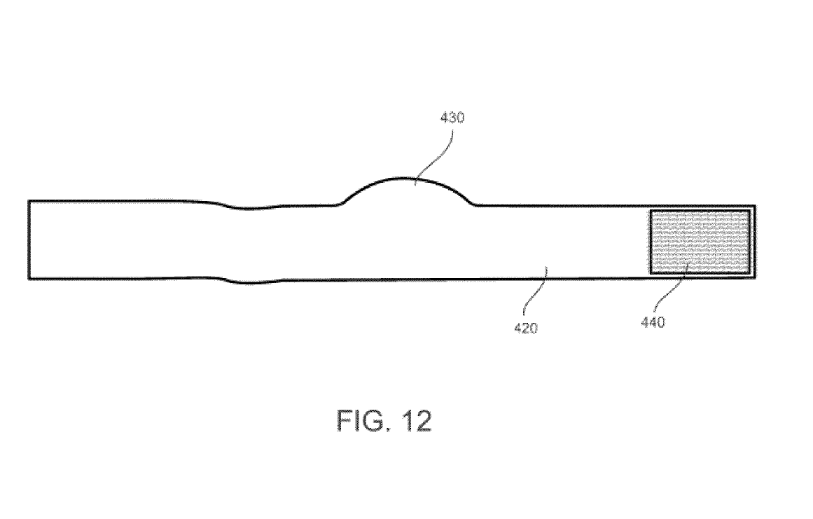

The garments disclosed herein may further include many different shapes and embodiments having a wide variety of uses. For example, these garments may be adapted for specific body parts and specific surgical procedures, which include, but are not limited, liposuction and surgical procedures involving the chin (e.g., chin implants), neck (e.g., a neck lift), etc. As shown in FIG. 10, another embodiment of the garment 300 may include the gel matrix 310 permanently positioned on an elongate, elastic bandage 320 and used to apply post-surgical compression and/or for thermal therapy and/or cold compression therapy. In this aspect, a hook fastener (not shown) may be attached one end of the elongate, elastic bandage and may fasten directly to another portion of the elongate, elastic bandage. Alternatively, the elongate, elastic bandage may include complementary hook and loop fasteners (not shown) positioned on opposite ends of the elongate, elastic bandage such that a this garment may be securely fastened to a wearer. As further illustrated in FIGS. 11 and 12, another embodiment of the garment 400 may include the gel matrix 410 being permanently positioned on an elongate, elastic bandage 420 and adapted for use on the chin, neck, or a combination thereof. In this aspect, FIGS. 11 and 12 particularly depict garment 400 useful, for example, in post-chin augmentation applications. The garment depicted in FIGS. 11 and 12 may include an arcuate shaped portion 430 of the gel and the elongate, elastic bandage adapted to cover a portion or substantially all of the wearer's chin.

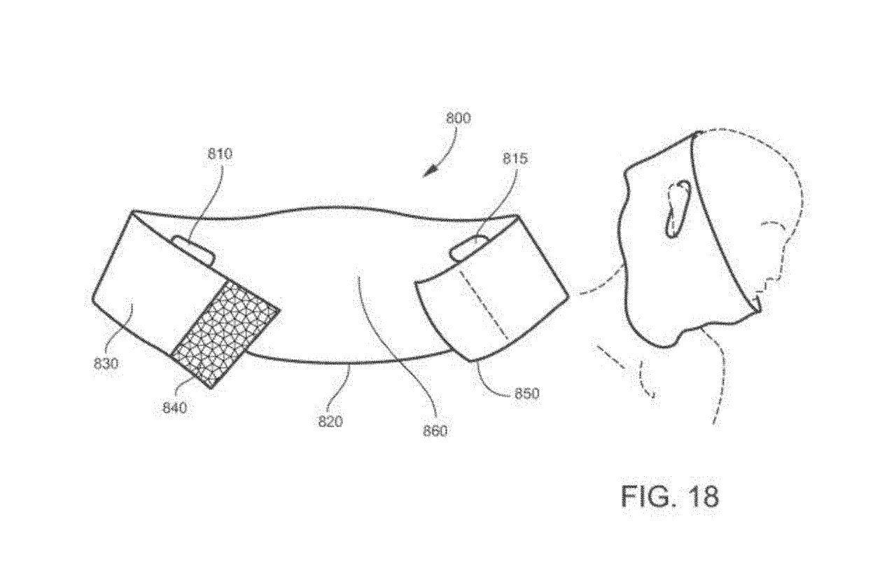

As shown in FIG. 18, in certain embodiments it is advantageous to provide a garment 800 having a substantial strength and sufficient width to provide robust neck and chin support post-surgical procedures involving the neck (e.g., a necklift, a necklift and chin augmentation, etc.). In this aspect, garment 800 is very similar to garment 400. For example, garment 800 includes the gel matrix layer 820 positioned on an elongate, elastic bandage 830. Garment 800 further includes complimentary hook and loop fasteners 840, 850 positioned on opposing ends of garment 800. However, garment 800 further includes earholes 810, 815 from which a wearer's ears may protrude through when wearing garment 800. Garment 800 provides robust support to wearer's neck following post-surgical neck procedures. In addition, portions 860 of the garment positioned in between earholes 810, 815 have a greater width than (i) portions of the garment extending between earhole 810 and hook or loop fastener 840 and (ii) portions of the garment extending between earhole 815 and hook or loop fastener 840. In other words, portions of the garment extending between earhole 810 and hook or loop fastener 840 and portions of the garment extending between earhole 815 and hook or loop fastener 840 are tapered relative to the width of garment 860 positioned between earholes 810, 815. These earholes 810, 815 and tapered features may be particularly preferred to provide robust support of a wearer's neck that further aids and expedites healing of the neck and/or chin following surgical procedures.

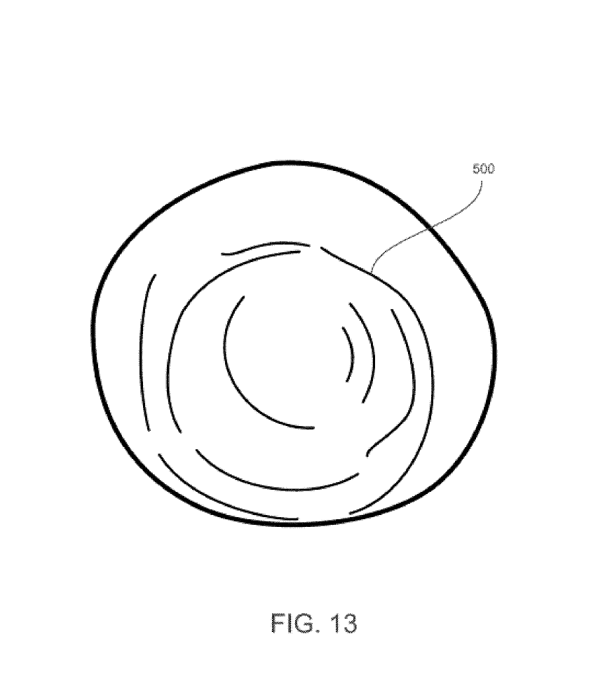

As illustrated in FIG. 13, in another embodiment, breast cup 500 includes the styrene-based polymeric gel adapted to conform to and cover a breast following a surgical procedure, which includes, but is not limited to breast augmentation, breast reconstruction, breast reduction, or combination thereof. In this aspect, the styrene-based polymeric gel may be molded (e.g., injection molded) to have the general shape and circumference of the breast. After placing this polymeric gel matrix over a user's breast, this gel may be further secured to a wearer with an elongate, elastic bandage being wrapped around the wearer's breast. In this aspect, the styrene-based polymeric gel may further include a thickness of 0.1 inches to 0.5 inches, 0.15 inches to 0.3 inches which advantageously ensures maximum reduction and/or prevention of post-surgical, excessive bruising, swelling, and edema associated with surgical procedures and further aids in the beneficial effects of thermal therapy and cold compression therapy. In certain aspects, breast cup 500 is packaged or included within a kit that further includes at least one elongate, elastic strap such as any of the above disclosed elongate, elastic straps, the strap being configured to hold breast cup 500 on a wearer's breast while applying adequate pressure to potentially reduce bruising, swelling, and/or edema at the surgical site.

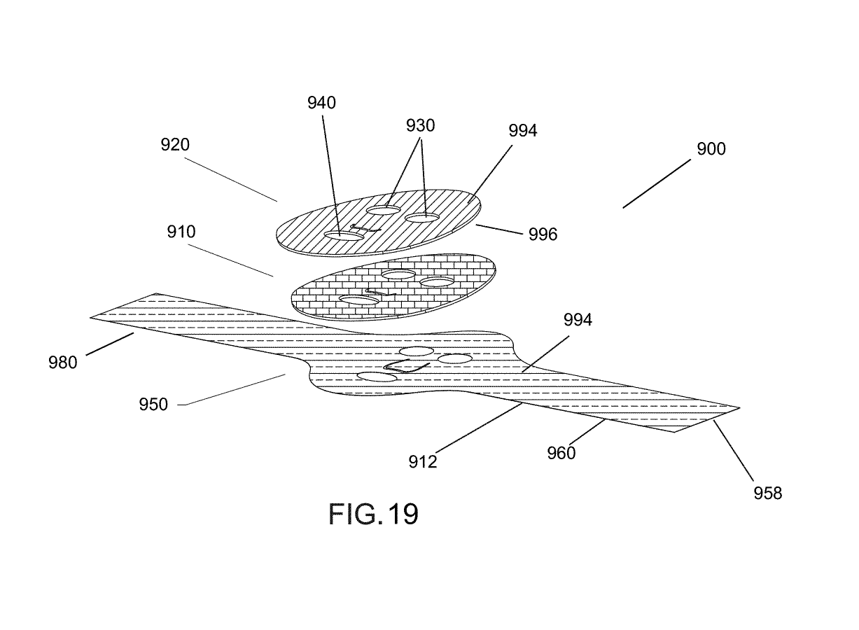

As illustrated in FIG. 19, another embodiment of the garment 900 may also include a thermoformable retainer layer 910 that is comprised of a polymer such as ethyl vinyl acetate (EVA) or polycaprolactone (PCL) or any admixture that is thermoformable at or below 60° C. This low temperature is desirable because it allows the garment to be conformed directly on an area of the user's skin. After being heated to the appropriate temperature, the thermoformable retainer layer 910 is applied to the user's face, depressed into the various contours, and allowed to cool until rigid, thus maintaining the geometry to which it is applied.

After the thermoformable layer has cooled, a PCM layer 920 is applied to the inside surface of the thermoformable layer that will contact the user's skin. The mask or other therapeutic device 920 has at least one eye hole 930 and at least one mouth hole 940. This configuration allows for the maximum amount of contact from the cosmetic or therapeutic mask to the user's face. The strapped fabric 950 is applied afterwards and used to hold the formable layer to the user's face. The strapped fabric has a first end 960 and second end 980 each having preferable having a fastener 958. Fasteners such as hook and loop, buttons, and other known fastening means can be used. The strapped fabric 950 also has at least one eye hole 930 and at least one mouth hole 940.

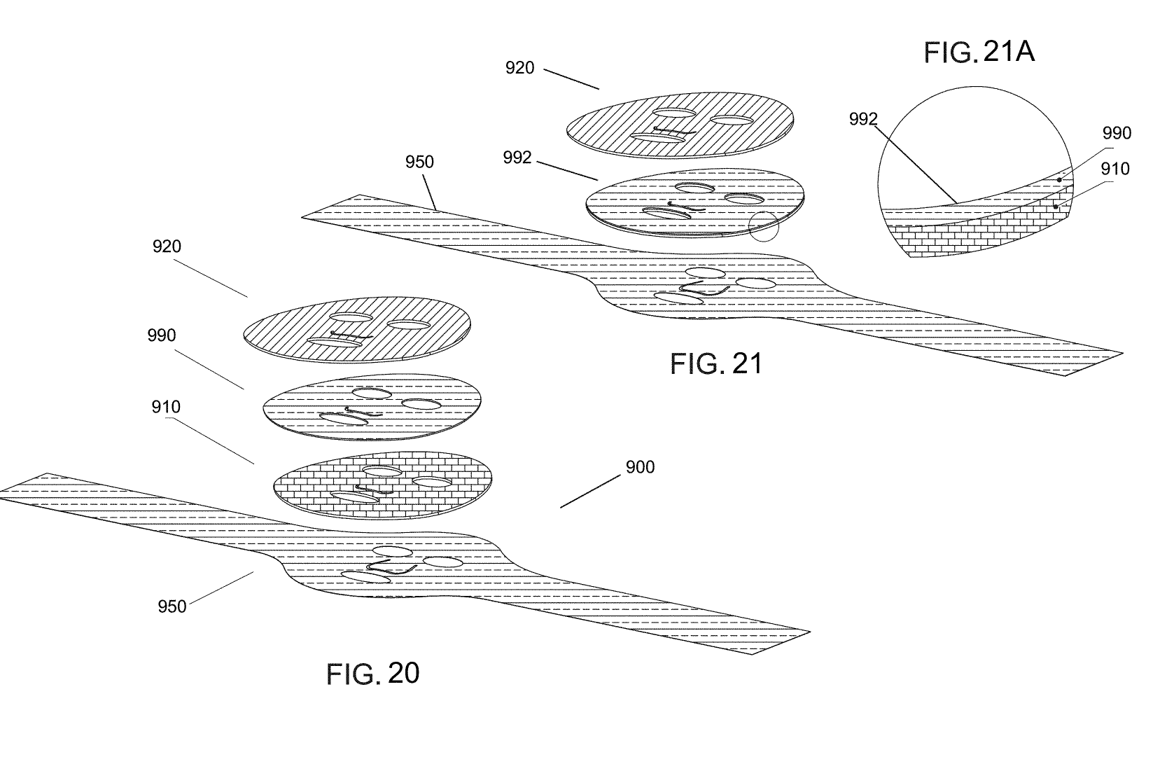

Illustrated in FIG. 20 is an exploded view of another embodiment of the present invention in which a layer of strapless fabric 990 is included in the retainer assembly. As shown in FIG. 21, the thermoformable retainer layer 910 has been bonded to a layer of strapless fabric 990 as a sub-assembly 992 of the garment 900 forming a composite. The thermoformable layer 910 also has at least one eyehole 930 and at least one mouth hole 940. Shown in FIG. 21A, the strapless fabric 990 is bonded to the thermoformable retainer layer 910 and is exposed towards the user's face with the cosmetic or therapeutic mask there between. The user first places the skin-engaging face 994 of the PCM layer 920 on their face and then places the heated sub-assembly 992 on the rear face 996 of the PCM layer 920. The internal face 998 of the strapped fabric 950 is then placed on top of the thermoformable retainer layer 910 which is then strapped into place using straps 912.

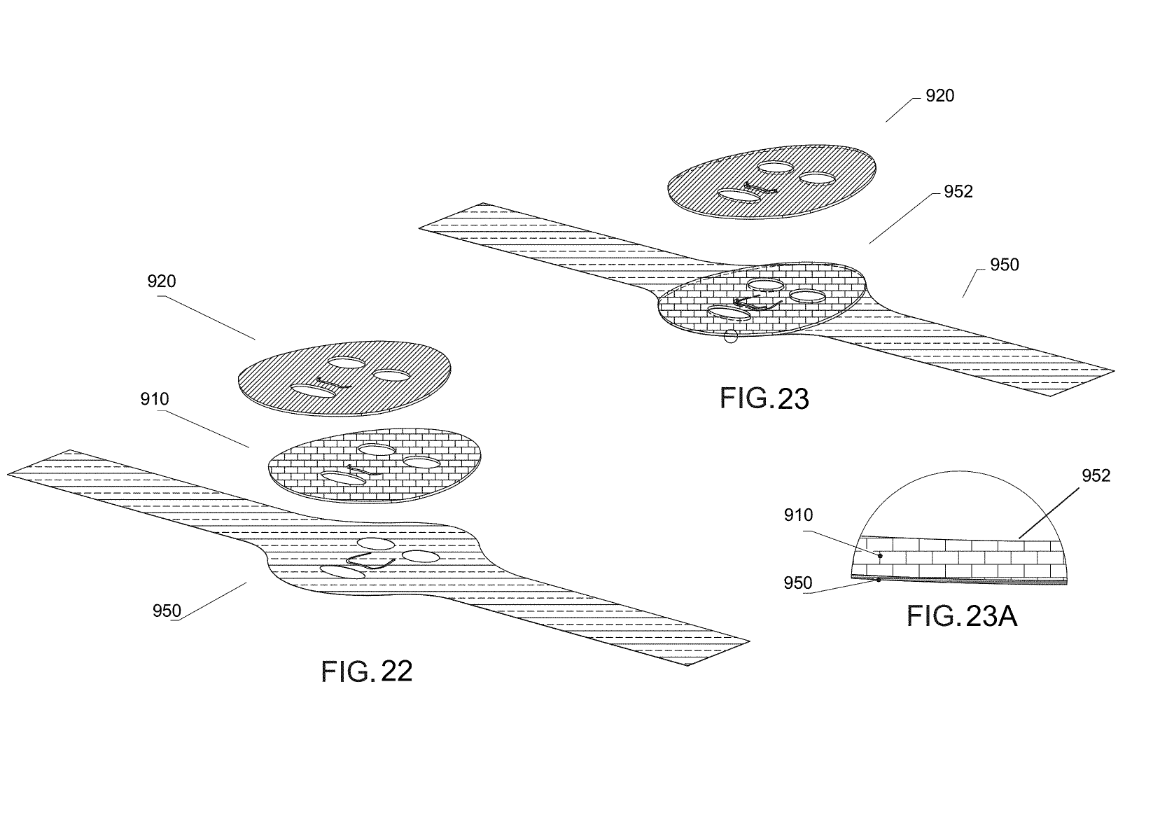

Illustrated in FIGS. 22 and 23 is another embodiment of the present invention in which the thermoformable retainer layer 910 is bonded to the strapped fabric 950 as a sub-assembly 952. The thermoformable retainer layer 910 is left exposed towards the user's face as seen in FIG. 23A. The PCM layer 920 is applied to the thermoformable layer and then to the user's face.

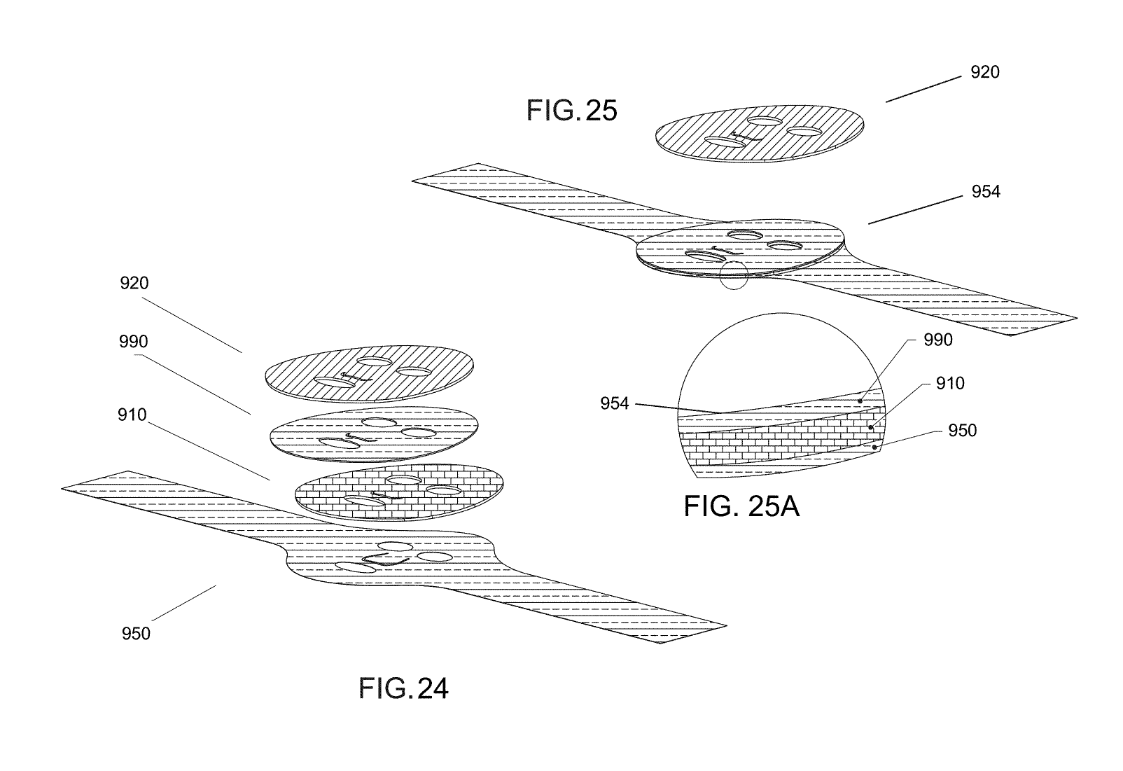

Illustrated in FIGS. 24 and 25 is another embodiment of the present invention in which the thermoformable retainer layer 910 is bonded on one side to strapped fabric 950 and on the other side to strapless fabric 990 as sub-assembly 954. The three component retainer assembly is heated, including strapless fabric 990, thermoformable retainer layer 910, and strapped fabric 950, to the transition temperature of the thermoformable retainer layer 910 and then pressed to the users face until the proper geometric contours are achieved. Afterwards, the PCM layer 920 is applied to the retainer assembly and applied to the user's face.

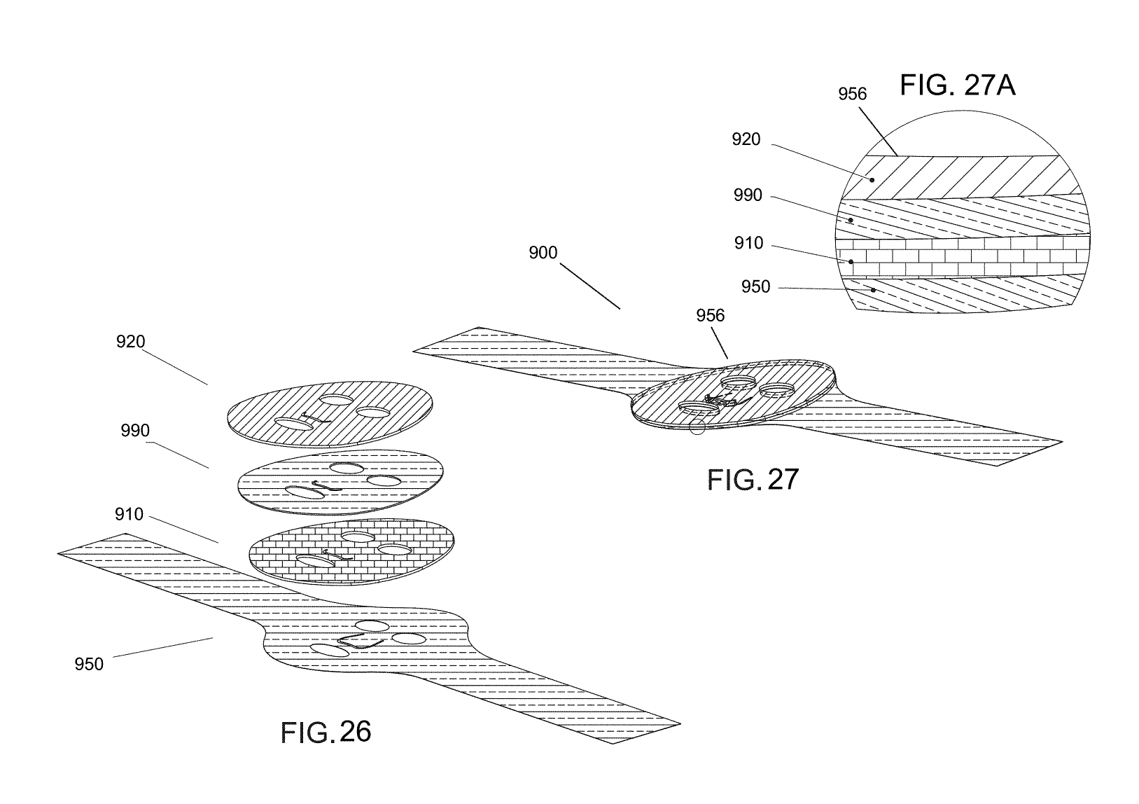

Illustrated in FIGS. 26 and 27 is another embodiment of the present invention in which one side of the thermoformable retainer layer 910 is bonded to strapped fabric 950 and the other is bonded to strapless fabric 990 as sub-assembly 956. In this embodiment the PCM layer 920 may be adhered, bonded, or fastened to the remaining surface of the strapless fabric. After heating to the transition temperature of the thermoformable material, the retainer assembly is depressed onto the user's face in order to form to the contours of the user's face.

Bonding between layers is generally achieved by heating the thermoformable layer beyond its heat deflection temperature and pressing the layer onto the layer with which it is desired to make a composite material. The layers may also be made into a composite by injecting or casting liquefied polymer onto the layer. It should be understood by those of ordinary skill in the art that similar techniques can be used to bond the various layers of the present invention together.

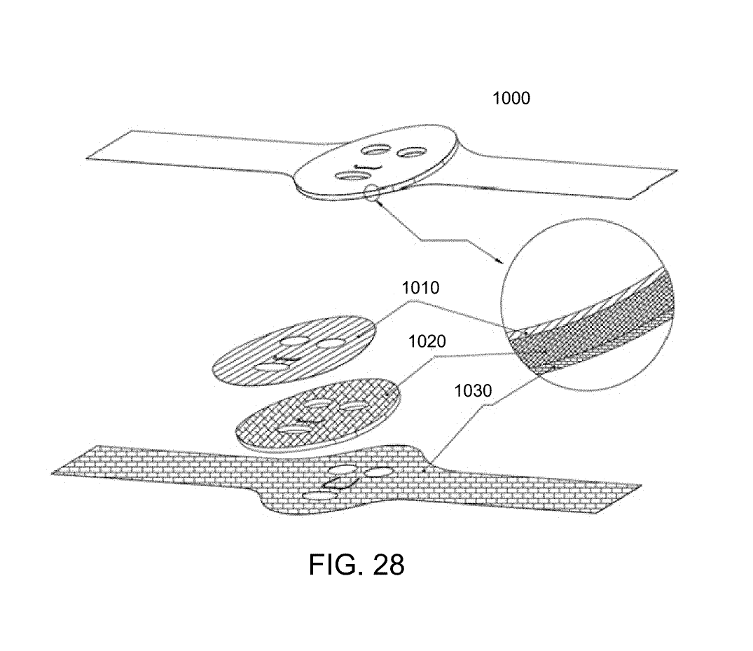

FIG. 28 shows a three layer embodiment of the garment 1000. These embodiments may or may not use the retainer described in FIGS. 19-27A. The garment 1000 is composed of up to three layers, one being a permeable layer 1010, which is located relative to a PCM layer 1020, which can be located relative to an optional securing fabric layer 1030. These three layers may be situated interchangeably depending on the user's needs. Furthermore, multiples of each layer may be used depending on the user's needs. For example, more than one permeable layer 1010 may be needed if the user requires more therapeutic agent to be transferred to their injured area.

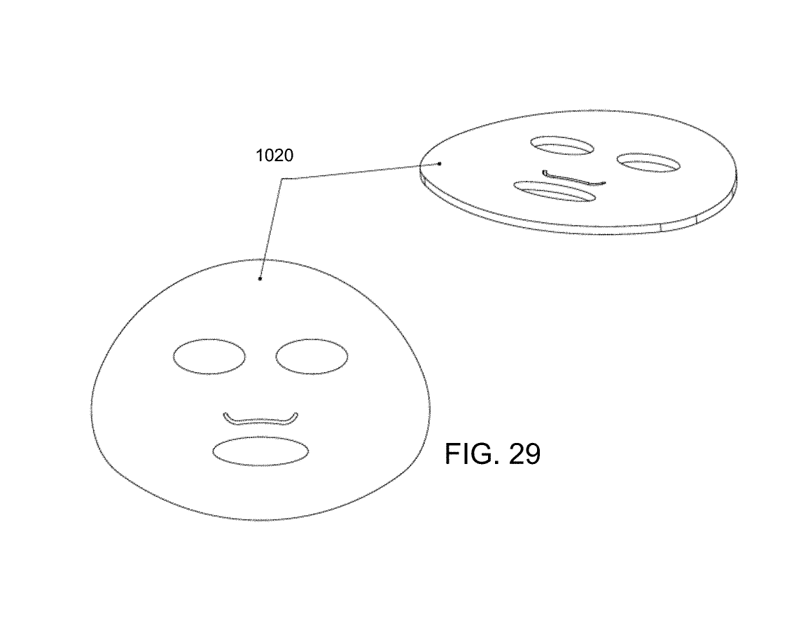

FIG. 29 shows the PCM layer 1020, which may or may not be located on or attached to the fabric layer 1030.

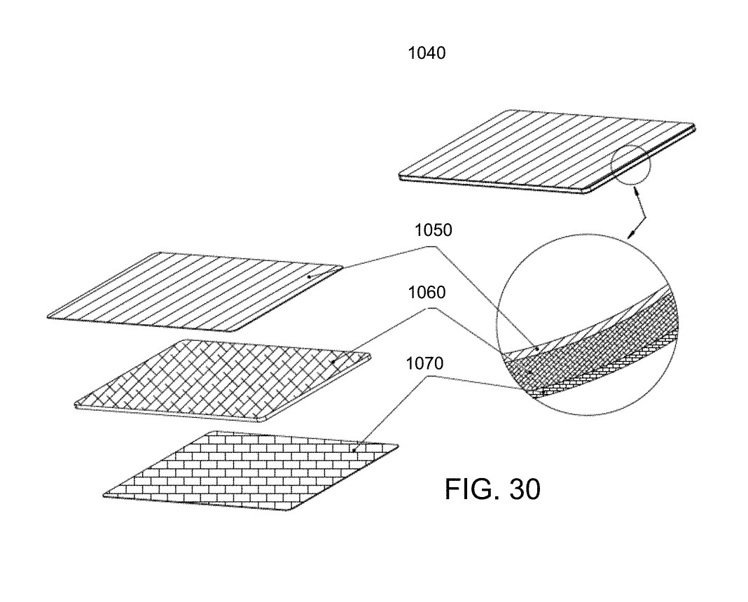

FIG. 30 shows an up to three layer embodiment of a garment 1040 meant to depict the garment 1040 in no particular shape. The garment 1040 can be any shape such as a mask, rectangular, square, or any other shape that allows for placement on a surface. The garment 1040 is composed of up to three layers, one being a permeable layer 1050, which is located relative to a PCM layer 1060, which is then located relative to a securing fabric layer 1070. These three layers may be situated interchangeably depending on the user's needs. Furthermore, multiples of each layer may be used depending on the user's needs as noted above. For example, more than one permeable layer 1050 may be needed if the user requires more therapeutic agent to be transferred to an injured area.

The polymeric gel layer 30 (or gel matrix layer 730 or PCM layer 920 depending on embodiment) can also include a plurality of reflecting fragments. These reflecting fragments are preferably distributed evenly throughout the polymeric gel layer 30. These fragments preferably comprise glitter or a similar material which reflect light. The average particle size of these fragments is preferably between 200-700 μm.

The garment 1040, when made with gel bonded to it, can be manufactured such that as few steps are needed to get a final product. First, the desired type of fabric is placed on around an aluminum substrate, although the substrate may be any similarly strong metal. A mold is then placed on the fabric. Heated gel is dispensed onto the portion of the fabric encompassed by the mold. The gel is then left to cure. This curing process generally takes 0.5 to 5 minutes depending on thickness. Once the gel has finished curing, the fabric and gel product is placed around a plastic substrate. This plastic substrate is between the portion of the fabric having gel on it and the portion without so that only one side of the fabric is die cut if features such as mouth holes, eye holes, etc. are desired.

In certain embodiments, the present invention may be configured to be placed within or onto a fabric, structure, garment, or any equivalent that is used for the purposes of contacting a user. The fabric, structure, garment, or any equivalent may be secured to the user by way of straps, strings, hook and loop fasteners, such as Velcro, magnets, rivets or any equivalent fastening means. Also, the fastening means may include techniques that allow the material to be situated within another material, such as a bag, towel, paper cloth, or any equivalent, which is then collectively secured or introduced to the user. For example, a user may place the present material into a damp towel, which is then situated to rest on her foot. Further, the securing of the disclosed invention may be accomplished without such fastening means, especially if the materials are configured to conform to the user's geometry or dimensions.

An alternative embodiment of the polymeric gel layer 30, one which can be applied independently of the other layers recited herein, can also be self-adhering and not require a retainer or straps. This functionality can be achieved by using plasticizers having a high viscosity together with a triblock copolymer of styrene end-blocks with ethylene or propylene or butylene mid-blocks or combinations thereof. Typical triblock copolymers include the general configuration A-B-A wherein each A is a glassy polymer end block segment of polystyrene and B is a elastomeric polymer center block segment of poly(ethylene-butylene), poly(ethylene-propylene) or poly(ethylene-ethylene-propylene). These polymers can take the form of poly(styrene-ethylene-ethylene-propylene-styrene) (also known as SEEPS), poly(styrene-ethylene-butylene-styrene) (also known as SEBS), poly(styrene-ethylene-propylene-styrene) (also known as SEPS), poly(styrene-butadiene-styrene) (also known as SBS), poly(styrene-isoprene-styrene) (also known as SIS), or variations thereof. A description of relevant polymers can be found in U.S. Pat. No. 6,552,109 to Chen.

Preferably, this embodiment of the polymeric gel layer 30 comprises between 2-30% by weight triblock copolymer and 35-98% by weight plasticizer having a viscosity between 30-300 centistokes (cst). Optionally, a fabric can be attached to the polymeric gel layer 30. Further, the polymeric gel layer 30 may include cosmetic or therapeutic agents in an amount between 0.1% and 35% by weight. What follows are examples of types of polymeric gel layer 30 that can be used in the present invention showing varying levels of adherence. In each of the examples below, the viscosity disclosed is the kinematic viscosity measured at 40° Celsius. The force required to pull the 180° peel test is measured in Newtons N and the substrate used for the peel test is preferably smooth stainless steel. The peel test is performed by laying the material on the substrate while leaving enough laying over the edge so that the material can be pulled as close to 180° from the surface of the substrate. The force for the material to break contact with the edge of the substrate is the force described below.

Each of examples 1-4 use a high molecular weight SEEPS gel while examples 5-7 use a low molecular weight SEEPS gel. The present invention is not limited to SEEPS gel products but can be any triblock copolymer.

### Example 1 (Non-Adhering Gel)

| Ingredient | Amount | |
|---|---|---|
| Polymer: SEEPS | 5% | by weight |
| Plasticizing oil | 95% | by weight |
| Viscosity = 10-14 cst | | |
| Stickiness | 0.6 | N |
| 180° peel test | | |

The gel of Example 1 generally does not adhere to a user's skin absent assistance from retainers or straps.

### Example 2 (Light Adherence Gel)

| Ingredient | Amount | |
|---|---|---|
| Polymer: SEEPS | 5% | by weight |
| Plasticizing oil | 95% | by weight |
| Viscosity = 63-70 cst | | |
| Stickiness | 2.2 | N |
| 180° peel test | | |

The gel of Example 2 will adhere to a user's skin but can be easily removed and may shift during use.

### Example 3 (Medium Adherence Gel)

| Ingredient | Amount | |
|---|---|---|
| Polymer: SEEPS | 5% | by weight |
| Plasticizing oil | 95% | by weight |
| Viscosity = 111-137 cst | | |
| 50% 63-70 cst | | |
| 50% 180-240 cst | | |
| Stickiness | 3.6 | N |
| 180° peel test | | |

The gel of Example 3 will adhere to a user's skin while maintaining placement without shifting during use. The gel of Example 3 may use a plasticizer that comprises a 50/50 mix of two different plasticizing oils. The viscosity of such a mixture can be found using the equation: v^{1/3}=xₐvₐ^{1/3}+x_{b}v_{b}^{1/3}. The plasticizing mixture should be comprised of plasticizing oils having a viscosity between 60-240 centistokes.

### Example 4 (Heavy Adherence Gel)

| Ingredient | Amount | |
|---|---|---|
| Polymer: SEEPS | 5% | by weight |
| Plasticizing oil | 95% | by weight |
| Viscosity = 180-240 cst | | |
| Stickiness | 4.3 | N |
| 180° peel test | | |

The gel of Example 4 is very adherent and may require additional effort to remove from a user's skin. The gel of Example 4 will not shift during use. As can be seen from these examples, adherence is a function of the viscosity of the plasticizing oil.

In certain embodiments, the present invention may be configured to form to a user's face, head, neck, upper or lower torso, feet, legs, or arms, or any combinations thereof. The user's specific needs for using the present invention will dictate its geometric form.

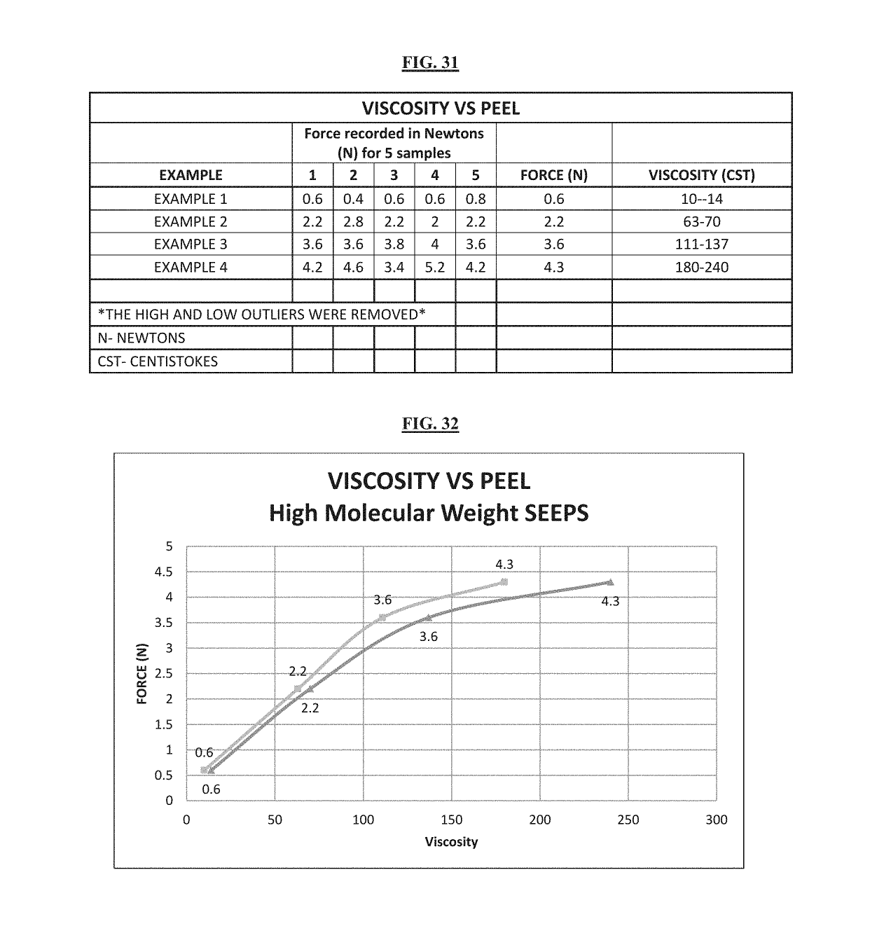

As seen in FIGS. 31 and 32, stickiness increases with the viscosity of the plasticizer, for high molecular weight SEEPS, rapidly first, from 0.6 N to 3.0 N, when going from a plasticizer with viscosity of 10 cst to one with viscosity of 100 cst then at a lower rate, from 3 N to 4 N, when going from a plasticizer with a viscosity of 100 cst to one with viscosity of 200 cst. The stickiness gain is reduced from 2.4 N for 90 cst increase in viscosity versus 1 N gain for 100 cst increase in viscosity.

Example 5, 6, and 7 use a low molecular weight SEEPS and exhibit lower stickiness than high MW SEEPS using the same plasticizing oils of example 2 3 and 4 respectively.

### Example 5 (SEEPS Low MW)

| Ingredient | Amount | |
|---|---|---|
| Polymer: SEEPS low MW | 5% | by weight |
| Plasticizing oil | 95% | by weight |
| Viscosity = 63-70 cst | | |
| Stickiness | 1 | N |
| 180° peel test | | |

### Example 6 (SEEPS Low MW)

| Ingredient | Amount | |
|---|---|---|
| Polymer: SEEPS low MW | 5% | by weight |
| Plasticizing oil | 95% | by weight |
| Viscosity = 111-137 cst | | |
| 50% 63-70 cst | | |
| 50% 180-240 cst | | |
| Stickiness | 1.26 | N |
| 180° peel test | | |

### Example 7 (SEEPS Low MW)

| Ingredient | Amount | |
|---|---|---|
| Polymer: SEEPS low MW | 5% | by weight |
| Plasticizing oil | 95% | by weight |
| Viscosity = 180-240 cst | | |
| Stickiness | 2.73 | N |
| 180° peel test | | |

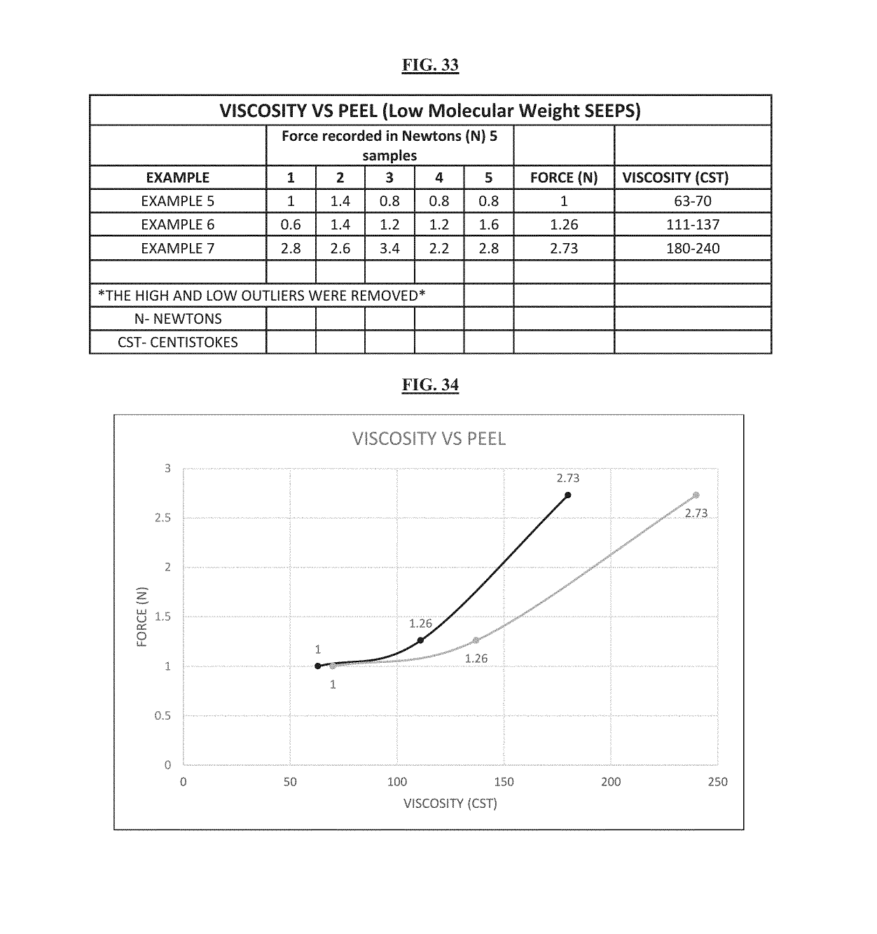

As it can be observed in FIGS. 31-34 for the high and low molecular weight SEEPS, the stickiness is greatly reduced when using low molecular weight SEEPS, when everything else is held constant, i.e. percent of polymer in plasticizing oil or type of plasticizing oil. More specifically the stickiness when using the same viscosity plasticizer is reduced when using low molecular weight versus high molecular weight SEEPS as follows: at 63 to 70 cst the stickiness is reduced from 2.2 N to 1 N, at 111-137 cst the stickiness is reduced from 3.6 N to 1.26 N, and at 180-240 cst the stickiness is reduced from 4.3 N to 2.73 N.

The present disclosure includes that contained in the appended claims, as well as that of the foregoing description. Although this invention has been described in its preferred form with a certain degree of particularity, it is understood that the present disclosure of the preferred form has been made only by way of example and that numerous changes in the details of construction and the combination and arrangement of parts may be resorted to without departing from the spirit and scope of the invention.

What is claimed is:

1. A self-adherent material comprising:
a triblock copolymer comprising polystyrene end blocks and a mid-block selected from the group consisting of polyethylene, polypropylene, polybutylene, polyisoprene, polybutadiene, and a mixture thereof; and
a plasticizing oil having a viscosity between 30 and 300 centistokes.

2. The self-adherent material of claim 1 wherein the material further comprises a fabric backing.

3. The self-adherent material of claim 1 further comprising at least one lipophilic cosmetic or therapeutic agent in a range between 0.1% and 35%.

4. The self-adherent material of claim 2 further comprising at least one lipophilic cosmetic or therapeutic agent in a range between 0.1% and 35%.

5. The self-adherent material of claim 1 wherein the plasticizing oil is selected from the group consisting of mineral oil, white oil, paraffinic oil, naphthenic oil, vegetable oil, a synthetic liquid oligomer such as a polybutene, a synthetic liquid oligomer such as polypropylene, and a synthetic liquid oligomer such as polyterpene oil.

6. The self-adherent material of claim 2 wherein the plasticizing oil is selected from the group consisting of mineral oil, white oil, paraffinic oil, naphthenic oil, vegetable oil, a synthetic liquid oligomer such as a polybutene, a synthetic liquid oligomer such as polypropylene, and a synthetic liquid oligomer such as polyterpene oil.

7. The self-adherent material of claim 1 wherein the triblock copolymer comprises 2-30% by weight of the self-adherent material.

8. The self-adherent material of claim 2 wherein the triblock copolymer comprises 2-30% by weight of the self-adherent material.

8. The self-adherent material of claim 1 wherein the plasticizing oil comprises 35-98% by weight of the self-adherent material.

9. The self-adherent material of claim 2 wherein the plasticizing oil comprises 35-98% by weight of the self-adherent material.
